(19) United States
(12) Patent Application Publication
(10) Pub. No.: US 2019/0150967 A1
(43) Pub. Date: May 23, 2019

(54) AVULSION FORCEPS

(71) Applicant: UNITED STATES ENDOSCOPY GROUP, INC., Mentor, OH (US)

(72) Inventors: Greg Haber, New York, NY (US); Keith R. John, Chardon, OH (US); John P. Winstanley, Madison, OH (US); Paul Martino, Solon, OH (US); Christopher J. Kaye, Middleburg Heights, OH (US)

(21) Appl. No.: 16/192,750

(22) Filed: Nov. 15, 2018

Related U.S. Application Data
(60) Provisional application No. 62/586,573, filed on Nov. 15, 2017.

(51) Int. Cl. A61B 17/29; A61B 17/00

(52) U.S. Cl. CPC A61B 17/29; A61B 17/00234; A61B 2017/2902; A61B 2017/00353; A61B 2017/2939; A61B 2017/2943

(57) ABSTRACT

A device for obtaining a tissue sample includes a spring sheath, an outer sheath, a base, a pair of jaws, and an actuator assembly. The outer sheath has a wall extending along a longitudinal axis. The base is connected to at least one of the spring sheath and the outer sheath. The pair of jaws is operatively connected to the base such that the pair of jaws can be moved between an open position and a closed position. Each jaw has a proximal portion and a distal portion. The actuator assembly includes a driver that extends through the base and engages the pair of jaws to move the pair of jaws between the open and closed positions. When the pair of jaws are in a fully opened position, the proximal portion of each jaw does not extend beyond the longitudinal axis of the wall of the outer sheath.

### CROSS-REFERENCE TO RELATED APPLICATIONS

The present application claims benefits and priority to U.S. Provisional Patent Application No. 62/586,573, filed on Nov. 15, 2017, the entire disclosure of which is incorporated herein by reference.

### FIELD OF THE INVENTION

The various embodiments relate to forceps in general and more particularly to avulsion forceps to be used endoscopically, having a pair of jaws, which can be opened and closed to grab and tear tissue.

### BACKGROUND INFORMATION

Conventional endoscopic forceps devices, such as biopsy forceps, typically contain jaws or forceps at the distal end. More specifically, these endoscopic forceps devices have an outer sheath, a base, at least two jaw components, and a driving mechanism. The proximal portion of the jaw components are connected to the base such that the jaw components can move between open and closed positions, and the driving mechanism is utilized to engage the proximal portion of the driving components to facilitate this movement. As the driving mechanism moves the jaw components from the closed position to the open position, the proximal end of the jaw components extend outward beyond a longitudinal axis of the wall of the outer sheath.

In some embodiments, conventional forceps devices are charged with an electric current to help facilitate removal of the tissue from a patient. Because the proximal ends of jaw components for conventional endoscopic forceps extend outward beyond a longitudinal axis of a wall of an outer sheath, the proximal ends of the jaw components, due to their intimate contact with current and their material properties, conduct current when the device is energized.

Improvements to avulsion forceps may be made by improving the design of the jaw components, such that, when in an open position, the proximal ends of the jaw components are not extending outward beyond a longitudinal axis of the outer wall of the sheath. Improvements of the avulsion forceps may also be made by insulating a majority of the length of the base and/or at least a portion of the proximal ends of the jaw components when the device is charged with electric current.

### SUMMARY OF THE INVENTION

An exemplary device for obtaining a tissue sample includes a spring sheath, an outer sheath, a base, a pair of jaws, and an actuator assembly. The outer sheath has a wall extending along a longitudinal axis. The base is connected to at least one of the spring sheath and the outer sheath. The pair of jaws is operatively connected to the base such that the pair of jaws can be moved between an open position and a closed position. Each jaw has a proximal portion and a distal portion, in which the proximal portion includes at least one arm having an internally extending piece, an externally extending projection, and a curved surface. The proximal portion of each jaw is connected to the base such that the curved external surface pivots about the base to move the pair of jaws between the open and closed positions. The actuator assembly includes a driver that extends through the base and is configured to engage the internally extended piece of each jaw to move the pair of jaws between the open and closed positions. When the pair of jaws are in a fully opened position, the proximal portion of each jaw does not extend beyond the longitudinal axis of the wall of the outer sheath.

Another exemplary device for obtaining a tissue sample includes a spring sheath, a dielectric sheath, a base, a pair of jaws, and an actuator assembly. The dielectric sheath at least partially surrounds the spring sheath. The base has a proximal portion and a distal portion, in which the proximal portion is connected to at least one of the spring sheath and the dielectric sheath. A length of the proximal portion of the base is greater than a length of the distal portion of the base. The pair of jaws is operatively connected to the distal portion of the base such that the pair of jaws can be moved between an open position and a closed position. The actuator assembly includes a driver that extends through the base and is configured to engage the pair of jaws to move the pair of jaws between the open and closed positions. The dielectric sheath surrounds the proximal portion of the base.

Another exemplary embodiment of a device for obtaining a tissue sample includes a spring sheath, an outer sheath, a base, a pair of jaws, an actuator assembly, and an insulating coating. The outer sheath has a wall extending along a longitudinal axis. The base is connected to at least one of the spring sheath and the outer sheath such that a distal portion of the base is not surrounded by the outer sheath. The pair of jaws is operatively connected to the base such that the pair of jaws can be moved between an open position and a closed position. Each jaw has a proximal portion and a distal portion, and the pair of jaws is connected to the base such that the proximal portion of each jaw pivots in an outward direction beyond the longitudinal axis of the wall of the outer sheath when the pair of jaws is in the open position. The actuator assembly has one or more drive wires that engage the pair of jaws to move the pair of jaws between the open and closed positions. The insulating coating at least partially covers at least one of the distal portion of the base, the one or more drive wires, and the proximal portion of the pair of jaws.

These and other aspects of the exemplary embodiments will become apparent from the following detailed description, taken in conjunction with the accompanying drawings, illustrating by way of example the principles of the various exemplary embodiments.

### BRIEF DESCRIPTION OF THE DRAWINGS

In order to facilitate a fuller understanding of the exemplary embodiments, reference is now made to the appended drawings. These drawings should not be construed as limiting, but are intended to be exemplary only.

FIG. 1 depicts a perspective view of an exemplary embodiment of an avulsion forceps assembly;

FIG. 2 depicts a side view of the avulsion forceps assembly of FIG. 1;

FIG. 3 depicts an exploded view of the avulsion forceps assembly of FIG. 1;

FIG. 4A depicts a side view of a distal end of the avulsion forceps assembly of FIG. 1;

FIG. 4B depicts a top view of the avulsion forceps assembly of FIG. 1;

FIG. 5A depicts a perspective view of an exemplary embodiment of a jaw component for the avulsion forceps assembly of FIG. 1;

FIG. 5B depicts a perspective view of the jaw component of FIG. 5A;

FIGS. 5C depicts a perspective view of another exemplary embodiment of a jaw component for the avulsion forceps assembly of FIG. 1;

FIGS. 5D depicts a perspective view of another exemplary embodiment of a jaw component for the avulsion forceps assembly of FIG. 1;

FIGS. 5E depicts a perspective view of another exemplary embodiment of a jaw component for the avulsion forceps assembly of FIG. 1;

FIG. 6 depicts a side view of a distal end of the avulsion forceps of FIG. 1, in which an exemplary embodiment of the jaw assembly is in a closed position;

FIG. 7 depicts a side view of a distal end of the avulsion forceps of FIG. 1, in which the jaw assembly of FIG. 6 is in a partially-open position;

FIG. 8 depicts a side view of a distal end of the avulsion forceps of FIG. 1, in which the jaw assembly of FIG. 6 is in another partially-open position;

FIG. 9 depicts a side view of a distal end of the avulsion forceps of FIG. 1, in which the jaw assembly of FIG. 6 is in a fully-open position;

FIG. 10 depicts a side view of a distal end of the avulsion forceps assembly of FIG. 1, in which the jaw assembly of FIG. 6 is moving from the open position to the closed position;

FIG. 11 depicts a side view of another exemplary embodiment of an avulsion forceps assembly, in which exemplary embodiments of jaw components of the avulsion forceps assembly are in the closed position; and

FIG. 12 depicts a side view of the avulsion forceps assembly of FIG. 11, in which the jaw components are in an open position.

### DETAILED DESCRIPTION OF EMBODIMENTS

The following description is intended to convey a thorough understanding of the embodiments by providing various embodiments and details involving an avulsion forceps device that is configured to minimize thermal injury during use on a patient. It is understood, however, that the invention is not limited to these specific embodiments and details, which are exemplary only. It is further understood that one possessing ordinary skill in the art, in light of known devices, systems and methods, would appreciate the use of the invention for its intended purposes and benefits in any number of alternative embodiments.

Generally speaking, the various exemplary embodiments of the avulsion forceps described herein have an outer sheath, a base, a pair of jaws connected to the base such that the jaws can be moved between open and closed positions, and an actuating assembly that is configured to move the jaws between the open and closed positions. The avulsion forceps can be configured such that, when the jaw are in an open position, a proximal end of the jaws can extend outward beyond a longitudinal axis of the wall of the outer sheath, or such that the proximal end of the jaws do not extend outward past a longitudinal axis of the wall of the outer sheath. Embodiments in which the proximal end of the jaws do not extend beyond the walls of the outer sheath can be advantageous because less protrusions are extending from the device that could inadvertently contact tissue of a patient during use of the device.

Certain embodiments of the avulsion forceps in which the proximal ends of the jaws do not extend beyond the walls of the outer sheath (when the jaws are in the open position) include a first jaw and a second jaw, in which each jaw has a proximal portion and a distal portion, the proximal portion of each jaw having at least one internally extending arm with a curved proximal surface and an angled inner surface. Each jaw also has one externally extending projection with a curved external surface. The jaws can be combined with any actuating assembly having a driver, in which the driver is configured to engage and open the jaws such that the proximal portions of the jaw do not extend beyond the walls of the outer sheath.

Both embodiments of avulsion forceps described above (i.e., embodiments in which the proximal ends of the jaws extend beyond a wall of the outer sheath and embodiments in which the proximal ends of the jaws do not extend beyond a wall of the outer sheath) can be charged with electric current to facilitate removal of tissue. For example, the avulsion forceps can be biopsy forceps that are charged with electric current for tearing the tissue. The biopsy forceps can receive steady electric current or short bursts (or impulses) of electric current. Impulses of electric current, rather than steady electric current, prevents electrical current from inducing injury to surrounding tissue. In some of these embodiments, tissue is removed by both the electric current provided to the biopsy forceps and mechanical force applied to the avulsion forceps assembly.

Various embodiments described herein include features that minimize the potential for thermal injury as a result of the electric charge. For example, in embodiments in which the proximal ends of the jaws do not extend beyond a wall of the outer sheath, the outer sheath can be a dielectric sheath, and the dielectric sheath surrounds a majority of the length of the base. In another example, in embodiments in which the proximal ends of the jaws do extend beyond a wall of the outer sheath, a majority of the base and/or at least a portion of the proximal ends of the jaws can be coated with an insulating coating that insulates the coated portions of the jaws.

Generally speaking, a method of using an exemplary embodiment of the jaw assembly for avulsion forceps to obtain a tissue sample includes the step of opening a pair of jaws by pushing at least one drive wire in a distal direction to cause a drive member to move in a distal direction and engage the pair of jaws.. A method of using an exemplary embodiment of the invention to obtain a tissue sample also includes closing the pair of jaws around a volume of tissue, and includes the steps of pulling the at least one drive wire in a proximal direction, to cause the jaws to move to a closed position and grasp a volume of tissue. The tissue can be torn or removed from the body.

The various exemplary embodiments generally described devices that have a pair of jaws that may be used to tear or pull a volume of tissue away from a greater volume of tissue. However, the various embodiments are not so limited. For example, the jaws can be hot biopsy jaws, and the device can be configured to cut a volume of tissue by supplying short bursts of current to the biopsy jaws and also applying a mechanical force to the biopsy jaws. In some embodiments, the jaw assembly can be used to cut or cauterize tissue, or to reveal additional tools such as a cauterization tip or a needle for injection purposes.

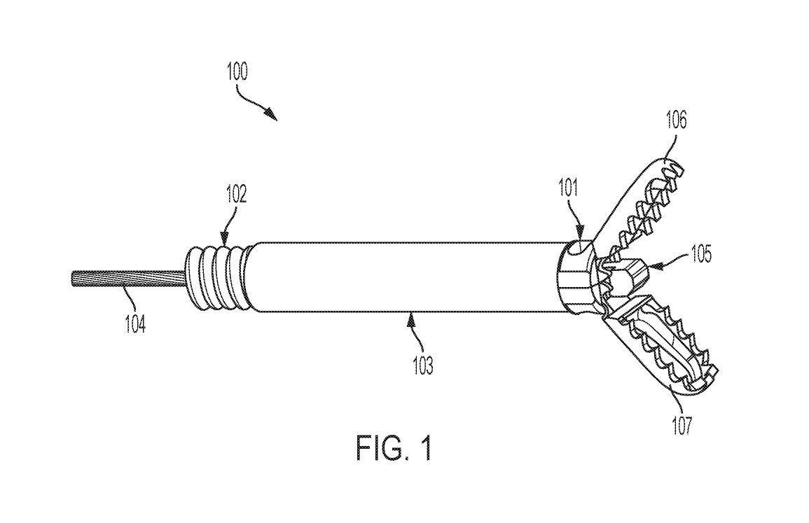

FIGS. 1 through 10 illustrate an exemplary embodiment of an avulsion forceps assembly. Referring to FIGS. 1 through 3, the forceps assembly 100 includes a base 101, a spring sheath 102, an outer sheath 103, and two jaw components 106, 107. The forceps assembly 100 can also include an actuating assembly having a drive cable 104 and a driver 105. The spring sheath 102 can be made of stainless steel or other conducting medium if the device is intended to have current run through it. The spring sheath 102 can be made of fluorinated ethylene propylene or other thermoset or thermoplastic polymer if electric current is not intended to run through it. In certain embodiments, the outer sheath 103 is a dielectric sheath that is configured to insulate at least some portions of the device 100 that are electrically charged. The dielectric sheath 103 surrounds the spring sheath 102 and may also surround a portion of the base 101. The dielectric sheath 103 may be made of a non-conductive material or include a non-conductive coating. The dielectric sheath 103 shields surrounding tissue from any electrical current running through the device.

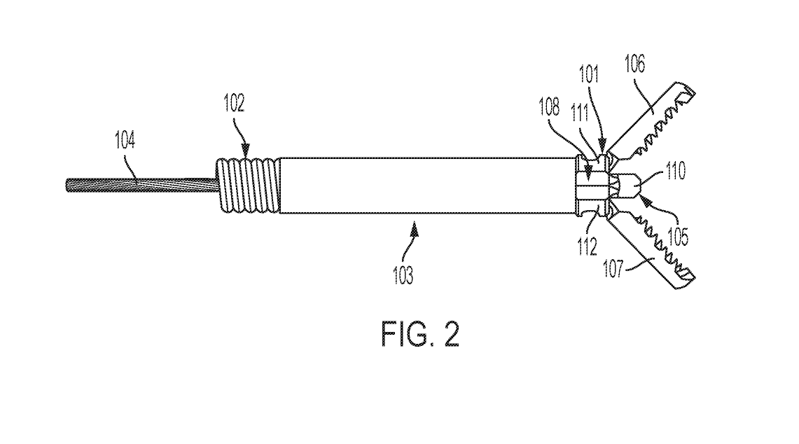

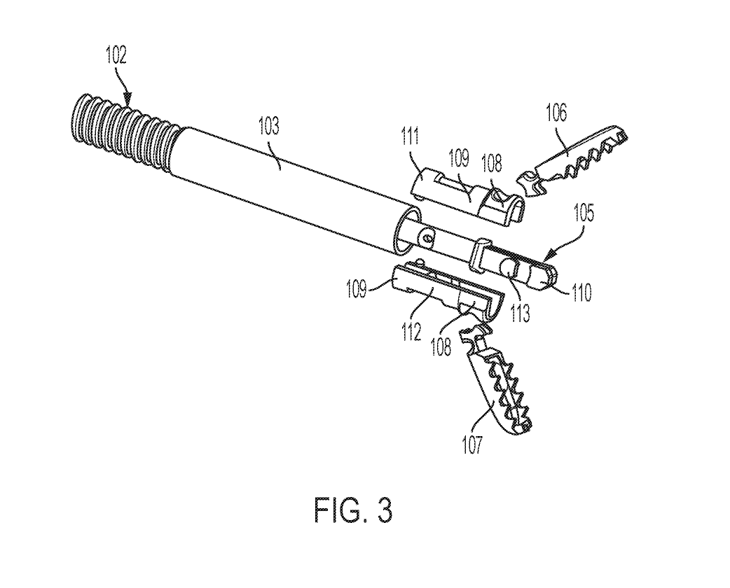

Referring to FIGS. 2 and 3, the base 101 includes a first base component 111 and a second base component 112 that are connected together to create a cylindrical shaft with a hollow interior. Each of the first and second base components 111, 112 have a distal portion 108 and a proximal portion 109. In other embodiments, the base 101 is a single piece having a hollow interior, and also has a distal portion 108 and a proximal portion 109. In certain embodiments, the length of the proximal portion 109 is greater than the length of the distal portion 108. The proximal portion 109 is connected to one of the spring sheath 102 and/or the outer sheath 103, and the distal portion 108 is operatively connected to the jaws 106, 107 such that the jaws are movable between the open and closed position. Referring to FIG. 2, in certain embodiments, a dielectric sheath 103 extends over the proximate portion 109 of the base 101 and is proximate to or abutting the distal portion 108 of the base 101. It is advantageous to have the dielectric sheath 103 extend close as possible to the jaws 106, 107 because the dielectric sheath can insulate more of the device 100 and prevent the transmission of electric current to surrounding tissue.

The drive cable 104 and the driver 105 may take any suitable form, such as, for example, any commercially available form that is capable of being used to engage the jaws 106, 107. In embodiments in which the driver is intended to have electric current run through it, the driver 105 can be made of stainless steel or other conducting medium, and, in embodiments in which the driver 105 is not intended to have electric current run through it, the driver 105 can be made of a non-conductive material. The driver 105, however, can be made of any suitable material that allows for the driver 105 to function as desired. The drive cable 104 is connected to the driver 105 such that movement of the drive cable 104 in the distal direction causes the driver 105 to move in the distal direction, and movement of the drive cable 104 in the proximal direction causes the driver 105 to move in the proximal direction. Referring to FIGS. 2 and 3, in certain embodiments, the driver 105 includes a distal engagement feature 110 and a proximal engagement feature 113 that are configured to engage the jaws 106, 107 to move the jaws between the open and closed positions.

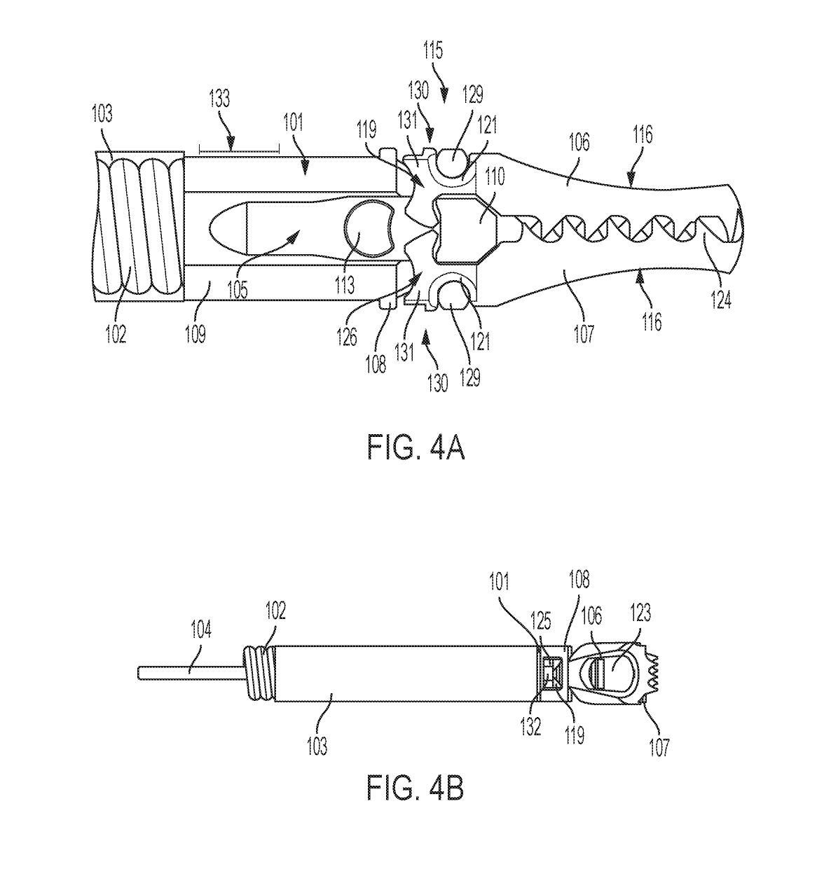

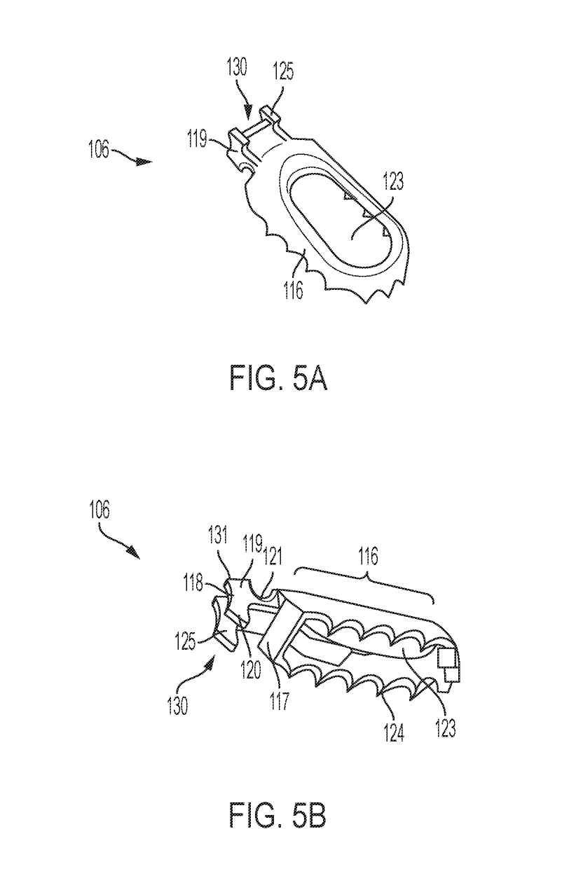

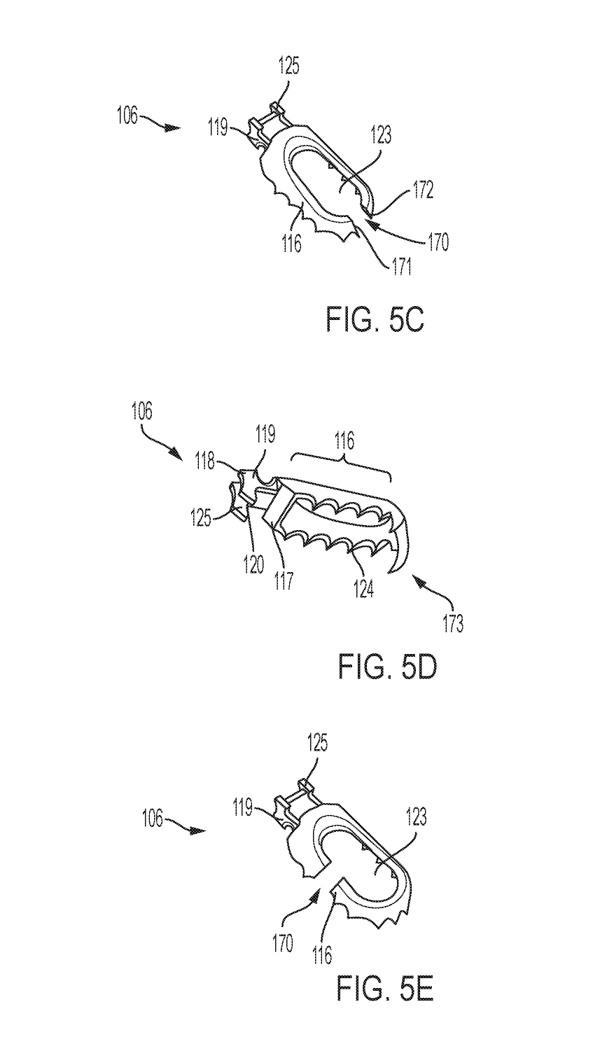

Referring to FIG. 4A, an exemplary embodiment of a jaw assembly 115 for the avulsion forceps 100 includes a first jaw 106 and a second jaw 107. Each jaw 106, 107 has a proximal portion 130 and a distal portion 116, in which the proximal portion 130 is connected to the base 101, and in which the distal portion 116 extends in a distal direction away from the base 101. The proximal portion 130 of both the first jaw 106 and the second jaw 107 includes at least one arm 119, 126 for connecting to the base 101. Referring to FIGS. 5A and 5B, in the illustrated embodiment, the first jaw 106 includes a first arm 119 and a second arm 125. While FIGS. 5A and 5B only show the first jaw 106 having two arms 119, 125, it should be understood that the second jaw 107 can also include two arms. It should also be understood that the jaws 106 can have a only one arm or more than two arms.

Referring to FIGS. 4A and 5A through 5B, the arms 119, 126, 126 can include an inwardly extending piece 120, an outward extending protrusion 131, a proximately located curved portion 118, and an outward curved portion 121. When the jaws 106, 107 are connected to the base 101, an outer edge 129 of the base 101 is disposed in the outward curved portion 121 of the arms 119, 125, 126 such that the jaws 106, 107 can pivot about the outer edge 129 of the base. The inwardly extending piece 120 extends into the hollow interior of the base 101 such that the engagement features 110, 113 of the driver 105 can engage the arms 119, 125, 126. The outward extending protrusion 131 extends into an opening 132 (FIG. 4B) at the distal portion 108 of the base 101 such that the outward extending protrusion 131 does not extend outward beyond the outer wall of the base 101 or the longitudinal axis 133 (FIG. 4A) of the wall of the dielectric sheath 103 when the jaws 106, 107 pivot between the open and closed positions. In certain embodiments, the openings 132 for each component 111, 112 of the base 101 are positioned 180 degrees from each other. It is advantageous to have the proximal portion 130 of the jaws 106, 107 not extend beyond the outer wall of the base 101 or the longitudinal axis 133 because less protrusions are extending from the device (as compared to a device in which the proximal end of the jaws do extend beyond the walls of the base and/or the sheath) that could inadvertently contact tissue of a patient during use of the device.

Still referring to FIGS. 4A and 5A through 5B, the distal portion 116 of the jaws 106, 107 include an angled surface 117 that is positioned to be engaged by the engagement feature 110 of the driver 105. The distal portion 116 can also include a plurality of teeth 124 on its surrounding edge, in which the teeth are positioned to interlock with the teeth of the other jaw 106, 107 when the jaws are in a closed position. Referring to FIGS. 4B and 5A through 5B, each jaw 106, 107 can have an opening 123 that provides a user with a window for targeting the tissue that is being removed. Each jaw 106, 107 has an elongate axis, and when assembled and in a closed position, the jaw arms extend in a proximal direction from the proximal end of the jaw, and then extend inward, towards a centrally located elongate axis of the jaw assembly. The jaws 106, 107 can be made of made of stainless steel or any other suitable commercially available material.

FIGS. 5C through 5E show alternative embodiments for the jaw 106, 107. Referring to FIG. 5C, in certain embodiments, the elongate distal portion has an opening 170 that provides extra space for tissue to be captured by the jaw assembly 115. In addition, the opening 170 includes edges 171, 172 that can help facilitate the tearing of the tissue and reduce surface area of tissue subjected to thermal injury during cautery. The opening 170 can be on one or both jaws 106, 107 such that a full or partial opening is created where each jaw is symmetrical, generating the notch on opposing sides during closure. Referring to FIG. 5D, in embodiments in which one of the jaws 106, 107 has an opening 170 (as shown in FIG. 5C), the other jaw 106, 107 can have an extended protrusion 173 that is aligned with the opening 170 such that, when the jaws 106, 107 are in a closed position, the extended protrusion 173 is disposed in the opening 170. The filling of the opening 170 with an extended protrusion 173 eliminates sharp ridges resulting from the edges 171, 172 of the opening 170, while in the closed position, which otherwise could be problematic as the jaw assembly moves through an endoscopic channel. The extended protrusion 173 also facilitates tearing of the tissue when the jaws 106, 107 are moved to the closed position. While the opening 170 and extended protrusion 173 are shown as being located at a distal most end of the jaws 106, 107, it should be understood that opening 170 and extended protrusion 173 can be located at any point along the elongate distal end of the jaws 106, 107. For example, referring to FIG. 5E, the opening 170 is shown on a side of the elongate distal portion of the jaws 106, 107. While FIG. 5E only shows an opening being located at the side of the elongate distal portion of the jaws 106, 107, it should be understood that the other jaw 106, 107 can have an extended protrusion 173 that aligns with the opening 170.

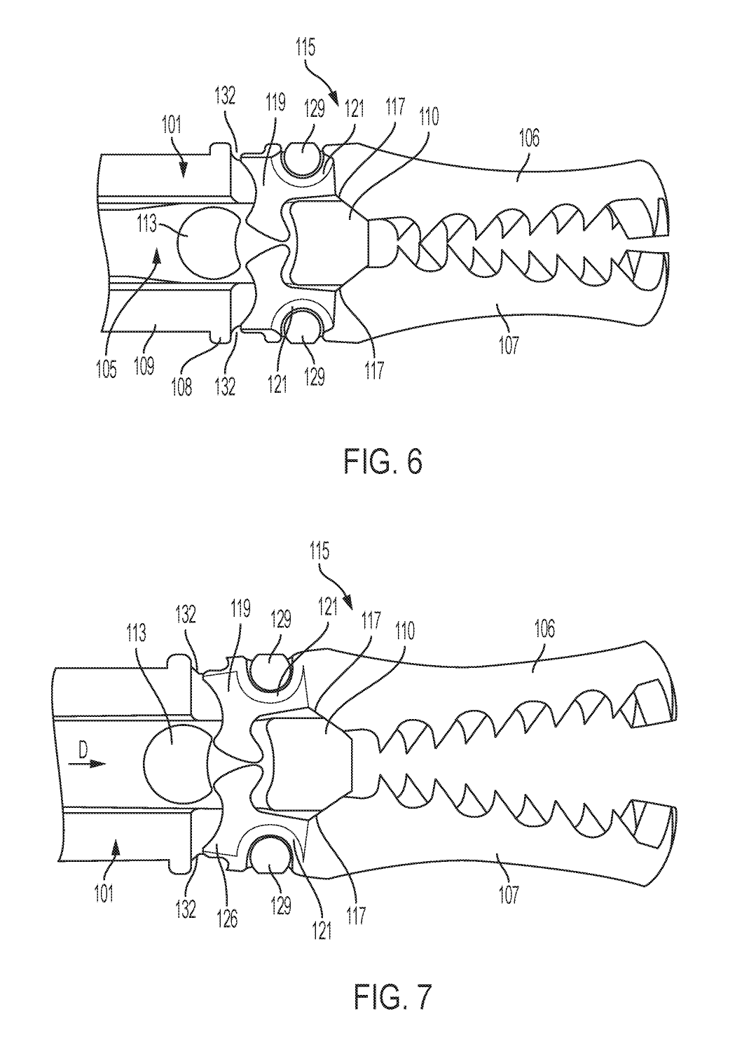

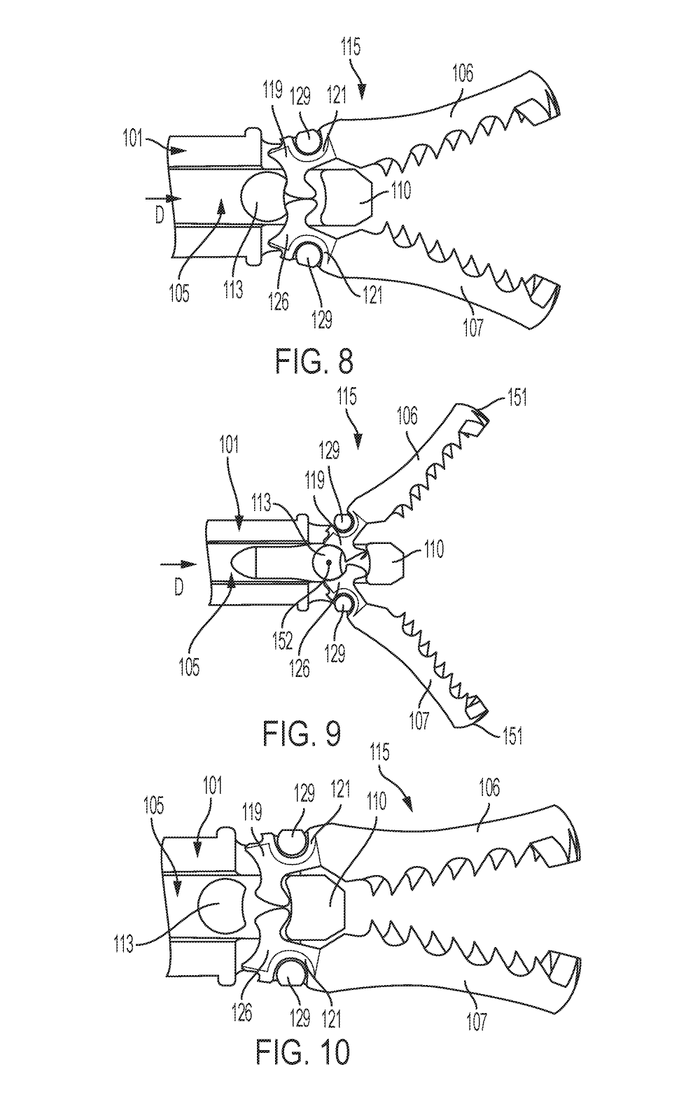

FIGS. 6 through 10 illustrate the driver 105 engaging the jaw assembly 115 to move the jaws 106, 107 between the open and closed positions. Referring to FIG. 6, in an initial position, the engagement feature 110 on the driver 105 is positioned to engage the angled surface 117 of each jaw 106, 107, and the proximal engagement feature 113 of the driver 105 is positioned to engage the inwardly extending piece 120 of the arms 119, 126. Referring to FIG. 7, the driver 105 is moved in the direction D (e.g., by a user forcing the drive wire 104 in the direction D), which causes the engagement feature 110 to engage the angle surface 117 of the jaws 106, 107 and the move the jaws 106, 107 to a first partially-opened position. Referring to FIG. 8, the continued movement of the driver 105 in the direction D causes the engagement feature 113 to engage the arms 119, 125, 126 of the jaws 106, 107 and cause the arms 119, 125, 126 to pivot about the edge 129 of the base 101 such that jaws 106, 107 move to a second partially-opened position. In the second partially-opened position, the engagement feature 110 is no longer engaging the jaws 106, 107 and is extended into the area between the distal portion 116 of each jaw member 106, 107.

Referring to FIG. 9, the driver 105 is moved in the direction D until the jaws 106, 107 are in the fully-opened position. In certain embodiments, when the jaws 106, 107 are in the fully opened position, the engagement feature 113 lock the jaws 106, 107 into position such that the jaws are not capable of opening any further. When the jaws 106, 107 are closed (as shown in FIG. 4A), the interior angle between the jaws is about zero (0) degrees, and when the jaws are fully opened (as shown in FIG. 9), the interior angle between the jaws is at a maximum, which can range from about eighty-five (85) degrees to about one hundred ten (110) degrees. When the jaws are open to the maximum degree permitted by the structure of the assembly, the distance between the distal-most point of each jaw is greater than the length of each jaw member 106, 107 from the distal-most point 151 to the intersection point 152 of the proximal ends of the jaws, which is approximately in alignment with the center of the engagement feature 113, should the longitudinal axes of the proximal portion of each jaw piece be extended in a proximal direction.

FIG. 10 illustrates the jaw as it begins to close again. In the illustrated embodiment, driver 105 is pulled in a proximal direction which causes the engagement feature 110 of the driver 105 to engage the inwardly facing arms 119, 126 t such that the arms 119, 126 are pushed in a proximal direction, thereby causing the curved portion 121 of the jaws to pivot around the edge 129 of the base 101. Thus the jaws pivot and close, and the angle between them decreases.

While the illustrated embodiment shows the exemplary embodiment of the driver 105 engaging the jaws 106, 107, it should be understood that any other suitable embodiment for a driver 105 that is capable of engaging the jaws 106, 107 to move the jaws between the open and closed positions can be used. While the illustrated embodiment shown in FIGS. 6 through 10 show one side of the jaw assembly 115, it should be understood that the other side of the jaw assembly can function in an identical manner.

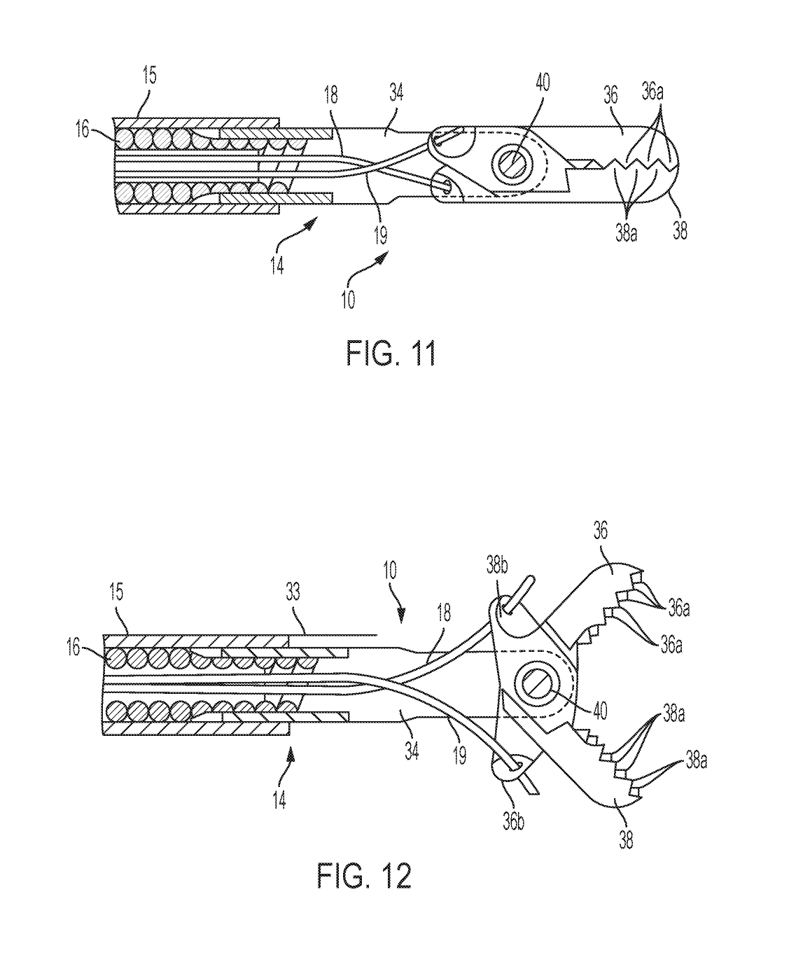

FIGS. 11 and 12 illustrate another exemplary embodiment of an avulsion forceps assembly 10. The forceps assembly 10 includes a base 34, a spring sheath 16, an outer sheath 15, two jaw components 36, 38, and an insulating coating 14. The forceps assembly 100 can also include an actuating assembly having one or more drive cables 18, 19. The spring sheath 16 can be made of stainless steel or other conducting medium if the device is intended to have current run through it. The spring sheath 16 can be made of fluorinated ethylene propylene or other thermoset or thermoplastic polymer if electric current is not intended to run through it. In certain embodiments, the outer sheath 15 is a dielectric sheath that is configured to insulate at least some portions of the device 10 that are electrically charged. The dielectric sheath 15 surrounds the spring sheath 16 and may also surround a portion of the base 34. The dielectric sheath 15 may be made of a non-conductive material or include a non-conductive coating. The dielectric sheath 15 shields surrounding tissue from any electrical current running through the device.

The base 34 has a proximal portion that is connected to at least one of the spring sheath 16 and/or the outer sheath 15, and a distal portion that is operatively connected to the jaws 36, 38 such that the jaws are movable between the open and closed position. In the illustrated embodiment, the jaws 36, 38 are connected the base 34 at a pivot point 40 such that the jaws can move between the open and closed positions. The drive cables 18, 19 are connected to the jaw components 36, 38 such that movement of the drive cables 18, 19 in the distal direction causes the corresponding jaw components 36, 38 to move to the open position (as shown in FIG. 12), and such that movement of the drive cables 18,19 in the proximal direction causes the corresponding jaw components 36, 38 to move to the closed position (as shown in FIG. 11). The drive cables 18, 19 may take any suitable form, such as, for example, any commercially available form that is capable of being used to engage the jaws 36, 38. In embodiments in which the drive cables 18, 19 are intended to be charged with electric current, the drive cables can be made of stainless steel or other conducting medium, and, in embodiments in which the drive cables 18, 19 are not intended to be charged with electric current, the drive cables can be made of a non-conductive material.

In the illustrated embodiment, each jaw 36, 38 has a proximal portion 36b, 38b and a distal portion having teeth 36a, 36b. The proximal portions 36b, 38b are connected to the base 34 and operatively connected to the drive wires 18, 19. This connection between the proximal portions 36b, 38b and the base 34 is configured such that movement of the drive wires 18, 19 in the distal direction causes the proximal portions 36b, 38b to move outward beyond the edge of the base 34 and beyond a longitudinal axis 33 of the wall of the outer sheath 15.

The insulating coating 14 is disposed over at least one of the base 34, drive wires 18, 19, and proximal portions 36b, 38b of the jaws 36, 38 to prevent these electrically charged components of the device 10 from inducing injury to surrounding tissue. In certain embodiments, the insulating coating 14 is applied to the portion of the base 34 that extends past the distal end of the outer sheath 15. In certain embodiments, the insulating coating 14 is applied to the portions of the drive wires 18, 19 and/or the portions of the proximal ends 36b, 38b that extend beyond the longitudinal axis 33 when the device 10 is in an open position. The insulating coating can be, for example, polytetrafluorethylene (PTFE), parylene, or any other suitable coating. It is advantageous to apply a coating to the device as described with the various embodiments above because these components are not protected by the dielectric sheath 15, and these components are in a position to contact surrounding tissue during use of the device 10.

The various embodiments are not to be limited in scope by the specific embodiments described herein. Further, although some of the embodiments have been described herein in the context of a particular implementation in a particular environment for a particular purpose, those of ordinary skill in the art should recognize that its usefulness is not limited thereto and that the various embodiments can be beneficially implemented in any number of environments for any number of purposes. Accordingly, the claims set forth below should be construed in view of the full breadth and spirit of the embodiments as disclosed herein. While the foregoing description includes many details and specificities, it is to be understood that these have been included for purposes of explanation only, and are not to be interpreted as limitations of the various embodiments. Many modifications to the embodiments described above can be made without departing from the spirit and scope of this description.

What is claimed is:

1. A device for obtaining a tissue sample, comprising:
a spring sheath;
an outer sheath having a wall extending along a longitudinal axis;
a base connected to at least one of the spring sheath and the outer sheath;
a pair of jaws operatively connected to the base such that the pair of jaws can be moved between an open position and a closed position, each jaw comprising a proximal portion and a distal portion, the proximal portion comprising at least one arm, wherein the arm comprises an internally extending piece, an externally extending projection, and a curved external surface, wherein the proximal portion of each jaw is connected to the base such that the curved external surface pivots about the base to move the pair of jaws between the open and closed positions;
an actuator assembly comprising a driver, wherein the driver extends through the base and is configured to engage the internally extended piece of each jaw to move the pair of jaws between the open and closed positions;
wherein the proximal portion each jaw does not extend beyond the longitudinal axis when the pair of jaws are in a fully opened position.

2. The device according to claim 1, wherein the distal portion of each jaw comprises an angled surface;

3. The device according to claim 2, wherein the driver comprises a distal engagement feature and a proximal engagement feature, and wherein the distal engagement feature engages the angled surface of the distal portion of each jaw and the proximal engagement feature engages the internally extending piece of each jaw to move the pair of jaws between the open and closed positions.

4. The device according to claim 1, wherein the at least one arm of each jaw comprises a first arm and a second arm.

5. The device according to claim 1, wherein the outer sheath is a dielectric sheath.

6. The device according to claim 1, wherein the base comprises a proximal portion and a distal portion, wherein the proximal portion of the base is connected to at least one of the outer sheath and the spring sheath, and wherein the distal portion of the base is connected to the pair of jaws.

7. The device according to claim 6, wherein the base comprises a first base component and a second base component, wherein a first jaw of the pair of jaws is connected to the first base component, and wherein a second jaw of the pair of jaws is connected to the second base component.

8. A device for obtaining a tissue sample, comprising:
a spring sheath;
a dielectric sheath at least partially surrounding the spring sheath;
a base comprising a proximal portion and a distal portion, wherein the proximal portion is connected to at least one of the spring sheath and the dielectric sheath;
a pair of jaws operatively connected to the distal portion of the base such that the pair of jaws can be moved between an open position and a closed position;
an actuator assembly comprising a driver, wherein the driver extends through the base and is configured to engage the pair of jaws to move the pair of jaws between the open and closed positions;
wherein the dielectric sheath surrounds a majority of a surface area of the base.

9. The device according to claim 8, wherein each jaw of the pair of jaws comprises a proximal portion and a distal portion, wherein the proximal portion comprises at least one arm, wherein the arm comprises an internally extending piece, an externally extending projection, and a curved external surface, wherein the proximal portion of each jaw is connected to the distal portion of the base such that the curved external surface pivots about the base to move the pair of jaws between the open and closed positions, wherein the distal portion comprises an angled surface.

10. The device according to claim 9, wherein the driver comprises a distal engagement feature and a proximal engagement feature, and wherein the distal engagement feature engages the angled surface of the distal portion of each jaw and the proximal engagement feature engages the internally extending piece of each jaw to move the pair of jaws between the open and closed positions.

11. The device according to claim 8, wherein the base comprises a first base component and a second base component, wherein a first jaw of the pair of jaws is connected to the first base component, and wherein a second jaw of the pair of jaws is connected to the second base component.

12. The device according to claim 8, wherein a length of the proximal portion of the base is greater than a length of the distal portion of the base, and wherein the dielectric sheath surrounds the entire proximal portion of the base.

13. The device according to claim 8, wherein at least one jaw of the pair of jaws has an elongate distal portion having an opening.

14. The device according to claim 13, wherein the at least one jaw has a first jaw that includes the opening and a second jaw that includes an extended protrusion, and wherein the extended protrusion of the second jaw is disposed in the opening of the first jaw when the par of jaws are in the closed position.

15. A device for obtaining a tissue sample, comprising:
a spring sheath;
an outer sheath having a wall extending along a longitudinal axis;
a base connected to at least one of the spring sheath and the outer sheath such that a distal portion of the base is not surrounded by the outer sheath;
a pair of jaws operatively connected to the base such that the pair of jaws can be moved between an open position and a closed position, each jaw comprising a proximal portion and a distal portion, wherein the pair of jaws is connected to the base such that the proximal portion of each jaw pivots in an outward direction beyond the longitudinal axis of the wall of the outer sheath when the pair of jaws are in an open position;
an actuator assembly comprising one or more drive wires that engage the pair of jaws to move the pair of jaws between the open and closed positions;
an insulating coating at least partially covering at least one of the distal portion of the base, the one or more drive wires, and the proximal portion of each of the pair of jaws.

16. The device according to claim 15, wherein a distal end of the one or more drive wires extends beyond the longitudinal axis of the wall of the outer sheath when the pair of jaws are in the open position.

17. The device according to claim 15, wherein the insulating coating comprises at least one of polytetrafluorethylene and parylene.

18. The device according to claim 14, wherein the insulating coating covers the entire distal portion of the base.

19. The device according to claim 14, wherein the insulating coating covers the entire portion of the pair of jaws that extends beyond the longitudinal axis of the wall of the outer sheath.

20. The device according to claim 14, wherein the outer sheath is a dielectric sheath.
